(19) United States
(12) Patent Application Publication
(10) Pub. No.: US 2026/0124079 A1
(43) Pub. Date: May 7, 2026

(54) REUSABLE URINARY INCONTINENCE SYSTEM

(71) Applicant: Marie Julianne Orr, Melrose, MA (US)

(72) Inventor: Marie Julianne Orr, Melrose, MA (US)

(21) Appl. No.: 18/939,806

(22) Filed: Nov. 7, 2024

(51) Int. Cl. A61F 13/49; A61F 13/15; A61F 13/505; A61M 1/00

(52) U.S. Cl. CPC A61F 13/49006; A61F 13/15268; A61F 13/505; A61M 1/71; A61F 2013/15146

(57) ABSTRACT

A urinary incontinence system that includes reusable mesh underwear configured to be worn by a patient. An absorption pad may rest in an opening in the underwear to collect urinary leakage. The system may further contain a suction tube connected to the absorption pad on its proximate end and to a suction cannister on the tube's distal end so as to direct the wearer's leakage from the absorption pad into the cannister. This cannister may then be emptied by the wearer at any time.

### BACKGROUND

Urinary incontinence, the involuntary leakage of urine, affects millions of individuals worldwide and can significantly impact their quality of life. It is a common condition, especially among older adults, women who have given birth, and individuals with certain medical conditions or surgeries. Various factors can contribute to incontinence, including weakened pelvic floor muscles, overactive bladder, and neurological disorders.

To manage urinary incontinence, a variety of products have been developed, such as absorbent pads, adult diapers, protective underwear, and external collection devices. These products aim to offer users discretion and protection against leakage, enabling them to carry out their daily activities with confidence. However, many of these solutions present challenges and limitations.

For instance, The Weisner Publication, DE Patent Application No. 2020/06,003,740, discloses an incontinence cloth has a liquid-tight outer covering, which has liquid permeable bags for retaining suction filters that is made up of an absorptive material. One of the filters is relocatably supported within the bags in such a manner that it is shifted from a reserve position which is provided in a hip area to a contact position by a strap or a string. This product offers a system where an absorbent pad must be replaced or repositioned on the wearer repeatedly after leakage incidents.

The Guangdong Patent, CN Patent No. 214,967,462, discloses medical equipment, namely, a male sex incontinence drainage device is disclosed, male sex incontinence drainage device includes man's pants and urethral catheterization cover, the anterior of man's pants is equipped with the elasticity guiding hole that supplies the male sex organ to pass with the corresponding position of patient's male sex organ, be equipped with the holding chamber in the urethral cover, the urethral cover on be equipped with the entrance point that the holding chamber is linked together, be equipped with all around of entrance point and paste the dish, be equipped with all around the elasticity guiding hole of man's pants and paste the installation position of dish cooperation location, urethral cover with the bottom of the relative one side of entrance point is equipped with the leakage fluid dram, the leakage fluid dram is connected with a catheter, the end-to-end connection of catheter has urine collection device. Adopt the utility model discloses technical scheme's male sex urinary incontinence drainage device can collect the urine of urinary incontinence patient's various positions, avoids lou urine, urine pollution clothing or the phenomenon that urine pollutes patient's skin to take place. However, the Guangdong Patent provides a cumbersome solution for male urinary incontinence, requiring the user's urethra to be in direct contact with a drainage tube.

Additionally, many existing products necessitate assistance from another person, are single-use and disposable, or require the wearer to carry a filled urinary bag in their clothing until they can find a place to dispose of the urine.

Conventional urinary incontinence devices fall short in delivering effective urinary leakage management while ensuring comfort, mobility, and versatility for active users. Many require assistance for removal or disposal, which compromises the wearer's dignity. Additionally, these “solutions” can be indiscreet, cumbersome, and unhygienic, promoting prolonged contact between urine and skin. Moreover, many products are only compatible with wearers of a sedentary lifestyle, rendering them ineffective the moment the wearer attempts any physical activity. This often results in embarrassment, irritation, and skin degradation, significantly detracting from the user's quality of life.

Accordingly, improved method and system for assisting urinary incontinence in patients are desired.

### SUMMARY OF THE INVENTION

This summary is provided to introduce a variety of concepts in a simplified form that is further disclosed in the detailed description of the embodiments. This summary is not intended for determining the scope of the claimed subject matter.

One embodiment of the present disclosure provides a urinary incontinence system, including a reusable mesh underwear configured to be worn by a patient. The underwear may have an opening for placement of a liquid absorption pad configured to fit into the opening. The liquid absorption pad may include a suction tube configured to have a proximate end to be positioned within the liquid absorption pad. The disclosed urinary incontinence system may include a suction cannister attached to a distal end of the suction tube for fluid collection.

Another embodiment of the present disclosure provides a method for assisting urinary incontinence in patients via a urinary incontinence system. The disclosed method may include: obtaining a reusable mesh underwear configured to be worn by a patient including an opening for placement of a liquid absorption pad, the liquid absorption pad configured to fit into the opening, a suction tube configured to have a proximate end to be positioned within the liquid absorption pad, and a suction cannister attached to a distal end of the suction tube for fluid collection. The disclosed method includes placing the reusable mesh underwear on the patient and draining the liquid absorption pad into the suction cannister.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a front view of a urinary incontinence system, according to the disclosed embodiments;

FIG. 2 illustrates a top view of the urinary incontinence system, according to the disclosed embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular systems and methods. Accordingly, the method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a system as an assembly comprising a combination of: (1) reusable mesh underwear; (2) a liquid absorption pad; and (3) a method for suctioning liquid from said absorption pad into a cannister (i.e., a suction tube connected on its proximate end to the absorption pad, which lies within the underwear and connected on its distal end to a cannister, which is connected to a suction mechanism via another tube), to: provide a comfortable and hygienic system to, for example, suction away one's urinary leakage from their body for holding in an adjacent cannister. By attaching the suction tubing and liquid holding cannister described herein to the liquid absorption pad within the reusable mesh underwear described herein, a system is formed for suctioning liquid away from the user's underwear into a cannister for disposal at a later time.

A urinary incontinence system is an assembly that includes a reusable mesh undergarment base, a liquid absorption pad, suction tubing, a liquid collection cannister, and a suction regulator. The liquid absorption pad is operatively connected to the reusable mesh undergarment. A liquid collection cannister is operatively connected to the absorption pad on one end via suction tubing and to a suction regulator on the other end via suction tubing.

The urinary incontinence system described herein addresses the unique challenges faced by urinary incontinence patients by focusing on comfortability, mobility, discretion, hygiene, and autonomy in the maintenance of a potentially rigid, conspicuous, and unsanitary medical condition, which have the following technical advantages, such as reusable underwear in connection with immediate urinary waste removal and remote urinary waste collection systems.

With respect to specialized application, the system described herein is uniquely designed for urinary incontinence patients, addressing a niche but critical need in the field.

With respect to comfort and mobility, the urinary incontinence system described herein is composed of reusable mesh underwear, thereby imparting a flexible and comfortable undergarment and permitting active and public lifestyles for urinary incontinence patients.

With respect to discretion, the urinary incontinence system described herein provides a remote liquid collection cannister, that is, not immediately adjacent to the user's underwear or groin area, thereby imparting discretion for the user as to conceal any otherwise bulky urinary incontinence maintenance system or apparatus.

With respect to hygiene, the urinary incontinence system described herein provides a method for immediate removal of urinary leakage from the user's underwear, thereby preventing skin damage and otherwise unsanitary exposure of the user's skin to the user's urine.

The urinary incontinence system described herein promotes user autonomy by enabling self-maintenance, particularly in the disposal and cleaning of the liquid collection cannister. This design supports the user's independence and dignity while providing discretion regarding their urinary incontinence.

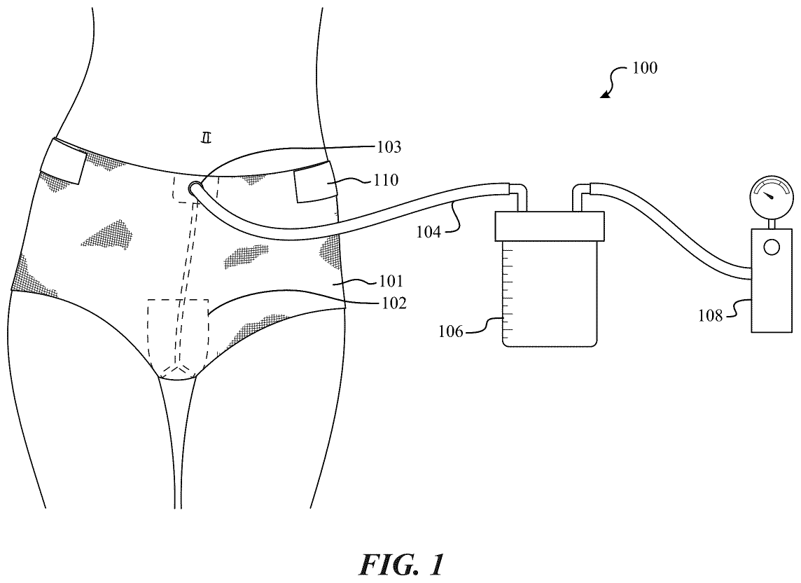

FIG. 1 illustrates a front view of a urinary incontinence system, according to the disclosed embodiments.

Referring to FIG. 1, the urinary incontinence system 100 implemented as a reusable mesh underwear 101 to be worn by, for example, a urinary incontinence patient. The urinary incontinence system 100 provides functionality for suctioning liquid (e.g., urine) from a liquid absorption pad 102 into a liquid collection cannister 106 connected to the liquid absorption pad 102 by a suction tube 104. In one embodiment, the liquid absorption pad 102 may be secured within the reusable mesh underwear 101, via an integrated receptor opening (i.e., a pocket that is not clearly shown) positioned within layers of the reusable mesh underwear 101 for facilitating an accurate positioning of the liquid absorption pad 102 within the reusable mesh underwear 101 for an effective absorption of patient's liquids. In one embodiment, the suction tube 104 may be attached to the liquid absorption pad 102 within the layers of the reusable mesh underwear 101. In one embodiment, the suction tube 104 may pass through a port 103 below the waistband of the reusable mesh underwear 101 for connection to the liquid absorption pad 102 within the layers of the reusable mesh underwear 101. In one embodiment, the location of the port 103 just below the waistband of the reusable mesh underwear 101 may be configured to allow for angle adjustment of the suction tube 104 on the exterior of the reusable mesh underwear, e.g., either right or left within the patient's pants. In one embodiment, the liquid collection cannister 106 may be attached to a suction regulator 108 via the suction tube 104. In one embodiment, the suction regulator 108 may have a compressor (not shown) configured to generate the suction functionality and subsequent draining of the urinary incontinence system 100. Thus, a liquid suction and collection functionality provides for hygienically collecting urine from the reusable mesh underwear 101. In one embodiment, the reusable mesh underwear 101 can be solid piece underwear, another with adhesive-based connections 110 at the waist to accommodate patients of all waist sizes.

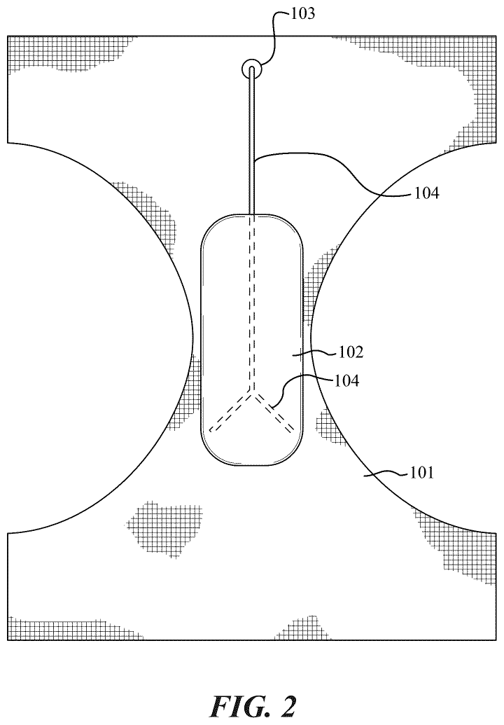

FIG. 2 illustrates a top view of the urinary incontinence system, according to the disclosed embodiments.

Referring to FIG. 2, the reusable mesh underwear 101 is shown here in an open pre-use position. In FIG. 2, the port 103 facilitates the passage of the suction tube 104 from the exterior of the reusable mesh underwear 101 for connection of the suction tube 104 to the liquid absorption pad 102 within the layers of the open reusable mesh underwear 101.

As discussed above with respect to FIG. 1, the reusable mesh underwear 101, advantageously, facilitates suctioning of the liquid (e.g., patient's urine) from the liquid absorption pad 102 via the suction tube 104. The liquid absorption pad 102 may be secured within the reusable mesh underwear 101 by an integrated receptor opening positioned within the layers of the reusable mesh underwear 101 for facilitating an accurate positioning of the liquid absorption pad 102 within the reusable mesh underwear 101 for an effective absorption of patient's liquids.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The foregoing is provided for purposes of illustrating, explaining, and describing embodiments of this disclosure. Modifications and adaptations to these embodiments will be apparent to those skilled in the art and may be made without departing from the scope or spirit of this disclosure.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

It will be appreciated by persons skilled in the art that the present embodiment is not limited to what has been particularly shown and described herein. A variety of modifications and variations are possible in light of the above teachings without departing from the following claims.

What is claimed is:

1. A urinary incontinence system, comprising:
a reusable mesh underwear configured to be worn by a patient, the underwear comprising an opening for placement of a liquid absorption pad;
the liquid absorption pad configured to fit into the opening;
a suction tube configured to have a proximate end to be positioned within the liquid absorption pad; and
a liquid collection cannister attached to a distal end of the suction tube for fluid collection.

2. The urinary incontinence system of claim 1, wherein the reusable mesh underwear is connected at a waist via adhesive-based connections, allowing adjustment for a patient's waist size.

3. The urinary incontinence system of claim 1, wherein the reusable mesh underwear comprises a port located below a waistband of the reusable mesh underwear configured to accommodate a passage of the suction tube for a connection within the liquid absorption pad.

4. The urinary incontinence system of claim 1, wherein a suction regulator is attached to a distal end of a suction tube having a proximate end to attached to the liquid collection cannister for liquid suction into the liquid collection cannister.

5. The urinary incontinence system of claim 1, wherein the liquid collection cannister is detachably connected to the distal end of the suction tube for disposal of a collected fluid.

6. The urinary incontinence system of claim 3, wherein the port is configured to allow for angle adjustment of the suction tube for patient manipulation.

7. A method for assisting urinary incontinence in patients via a urinary incontinence system, comprising:
obtaining the reusable mesh underwear configured to be worn by a patient, comprising:
an opening for placement of a liquid absorption pad;
the liquid absorption pad configured to fit into the opening;
a suction tube configured to have a proximate end to be positioned within the liquid absorption pad; and
a liquid collection cannister attached to a distal end of the suction tube for fluid collection; and
placing the disposable mesh underwear on the patient.

8. The method of claim 9, further comprising activating a suction of the fluid from the liquid absorption pad through the suction tube.
